(19) United States
(12) Patent Application Publication
(10) Pub. No.: US 2006/0264635 A1
(43) Pub. Date: Nov. 23, 2006

(54) Process for the preparation of moxifloxacin hydrochloride

(76) Inventors: Chava Satyanarayana, Secunderabad (IN); Gorantla Seeta Ramanjaneyulu, Secunderabad (IN); Vasireddy Umamaheswara Rao, Hyderabad (IN); Dammalapati Venkata Lakshmi Narasimharao, Hyderabad (IN)

Correspondence Address: SCHWEITZER CORNMAN GROSS & BONDELL LLP, 292 MADISON AVENUE - 19th FLOOR, NEW YORK, NY, 10017, US

(21) Appl. No.: 10/567,131

(22) PCT Filed: Aug. 5, 2004
(86) PCT No.: PCT/IN04/00233

§ 371 Date: Feb. 7, 2006

(30) Foreign Application Priority Data
Aug. 5, 2003 (IN) 638/CHE/2003
Aug. 5, 2003 (IN) 639/CHE/2003

(51) Int. Cl. C07D 471/02

(52) U.S. Cl. 546/113

(57) ABSTRACT

The present invention relates to an improved process for the preparation of Moxifloxacin hydrochloride from the ethyl 1-cyclopropyl 6,7-difluoro-8-methoxy-4-oxo-1,4-dihydro-3-quinolinecarboxylate through a novel intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-0³,0⁴)bis(acyloxy-0) borate.

The present invention relates to a process for preparation of Moxifloxacin hydrochloride, using a novel intermediate namely (4aS-Cis)-(1-cyclopropyl-7-(2,8-diazabicyclo[4.3.0]non-8-yl)-6-fluoro-8-methoxy-4-oxo-1,4-dihydro-3-quinolinecarboxylic acid-O³,O⁴)bis(acyloxy-O) borate.

### BACKGROUND OF THE INVENTION

Moxifloxacin Hydrochloride namely (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid hydrochloride has the formula

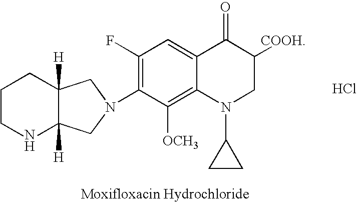

Moxifloxacin is a fluoroquinolone broad spectrum antibacterial particularly against Gram-positive bacteria significantly better than those of Sparfloxacin and Ciprofloxacin that was disclosed in EP No 350,733 and EP No 550,903. Moxifloxacin has activity against Gram-negative and Gram-positive organisms, including Streptococcus pneumonia, Staphylococcus aureus, Pseudomonas aeruginosa, particularly against the respiratory disease-causing pathogens like Mycoplasma pneumonia, Mycobacterium tuberculosis, Chlamydia pneumoniae and the activity shown to be unaffected by B-lactamases.

U.S. Pat. No 5,157,117 discloses (1-cyclopropyl-6,7-difluoro-8-methoxy-4-oxo-1,4-dihydro-3-quinoline carboxylic acid-O³,O⁴)bis(acyloxy-O) borate and a process for its preparation by reacting ethyl-1-cyclopropyl-6,7-difluoro-8-methoxy-4-oxo-1,4-dihydro-3-quinoline carboxylate with Boric acid and acetic anhydride in presence of zinc chloride and its conversion to Gatifloxacin hydrochloride.

Hydrates of Moxifloxacin hydrochloride known are the anhydrous and monohydrate. U.S. Pat. No. 5,849,752 discloses the monohydrate of Moxifloxacin hydrochloride and its preparation by treating the anhydrous crystalline form with ethanol/water mixtures.

The prior art disclosed in European Patent No's EP 350,733, EP 550,903 and EP 657,448 discloses the preparation of Moxifloxacin hydrochloride involving the condensation of 1-cyclopropyl-6,7-difluoro-8-methoxy-4-oxo-1,4-dihydro-3-quinoline carboxylic acid or its esters with (S,S) 2,8-Diaza bicyclo[4.3.0]nonane in presence of a base and its conversion to hydrochloride at higher temperatures leading to the desired Moxifloxacin along with its positional isomer namely (4aS-Cis)-1-cyclopropyl-6-(2,8-diazabicyclo[4.3.0]non-8-yl)-7-fluoro-8-methoxy-4-oxo-1,4-dihydro-3-quinoline carboxylic acid as a major impurity. As the impurity and the Moxifloxacin are positional isomers they are difficult to separate. Purification of Moxifloxacin to remove this isomer results in lower yields thereby increasing the product cost. Similarly methods described in the prior art involves the preparation of Moxifloxacin and then its conversion to its hydrochloride thereby incorporating an additional step in the manufacturing process also leading to lowering of yields.

It is a long felt need of the industry to provide high yielding and cost effective processes for the preparation of Moxifloxacin hydrochloride.

### SUMMARY OF THE INVENTION

The main object of the present invention is to provide a high yielding and cost effective process for the preparation of Moxifloxacin hydrochloride.

Another object of the invention is to provide a process for the preparation of Moxifloxacin hydrochloride without the additional step of isolation of Moxifloxacin.

Another object of the invention is to explore other hydrates of Moxifloxacin hydrochloride.

Another object of the invention is to provide the fingerprinting of Moxifloxacin hydrochloride pseudohydrate prepared by the invented process.

Another object of the invention is to provide a process for the conversion of Moxifloxacin hydrochloride pseudohydrate to Moxifloxacin hydrochloride monohydrate.

Another object of the invention is to provide a process for the preparation of the novel intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate and its use in the preparation for Moxifloxacin hydrochloride.

Another object of the invention is to provide fingerprinting of the novel intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate using NMR, IR and x-ray diffraction analysis.

Another object of the invention is to provide a process for the preparation of (1-cyclopropyl-6,7-difluoro-8-methoxy-4-oxo-1,4-dihydro-3-quinoline carboxylic acid-O³,O⁴)bis(acyloxy-O)borate without using the catalyst and its use for the preparation of Moxifloxacin hydrochloride.

Accordingly, the present invention relates to a method for the preparation of Moxifloxacin hydrochloride from the ethyl 1-cyclopropyl-6,7-difluoro-8-methoxy-4-oxo-1,4-dihydro-3-quinolinecarboxylate through novel intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate. The reaction of ethyl1-cyclopropyl-6,7-difluoro-8-methoxy-4-oxo-1,4-dihydro-3-quinoline carboxylate with boric acid and acetic anhydride without using any catalyst gives(1-cyclopropyl-6,7-difluoro-8-methoxy-4-oxo-1,4-dihydro-3-quinoline carboxylic acid-O³,O⁴)bis(acyloxy-O)borate which on condensation in presence of a base(s) with (S,S)-2,8-Diazabicyclo[4.3.0]nonane in organic polar solvent results the novel intermediate (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O) borate. This intermediate is reacted with hydrochloric acid in presence of solvent to give Moxifloxacin hydrochloride pseudo hydrate. The Moxifloxacin hydrochloride pseudohydrate is converted into Moxifloxacin hydrochloride monohydrate by treating with hydrochloric acid in presence of ethanol.

The reaction scheme is given below:

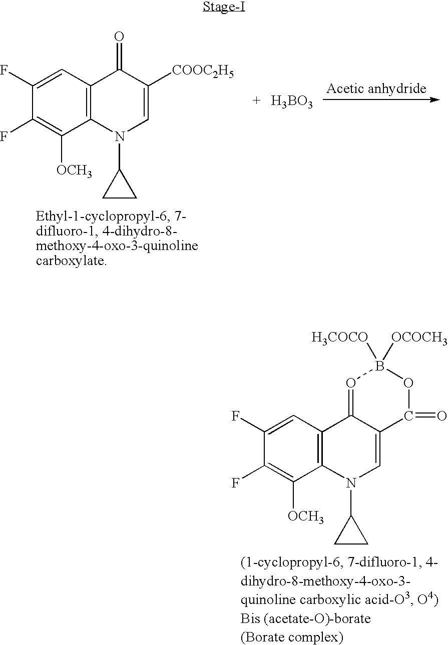

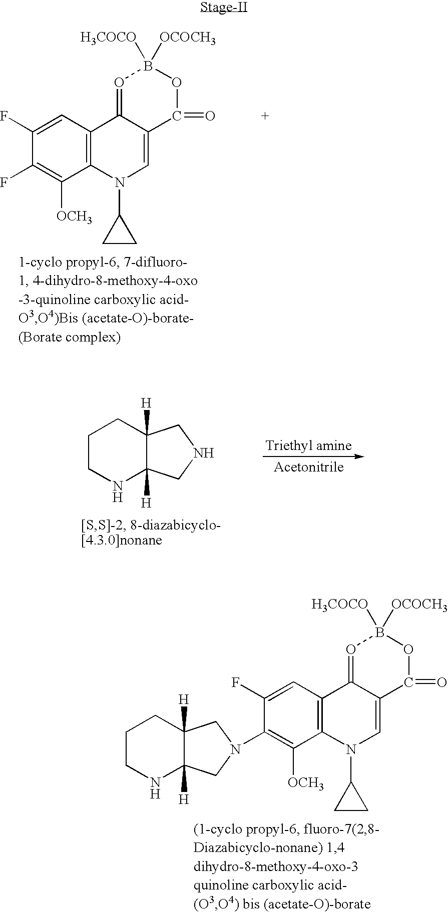

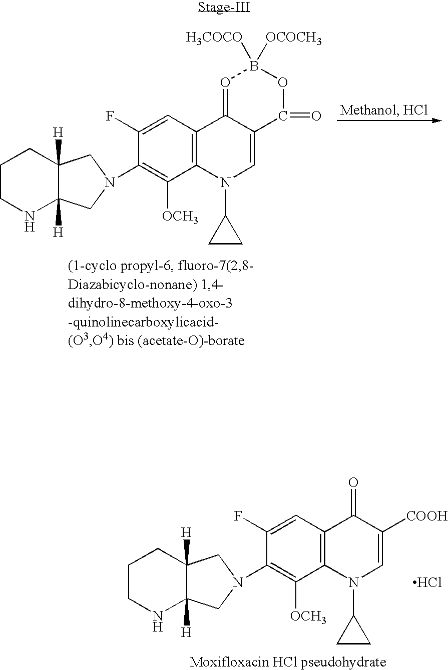

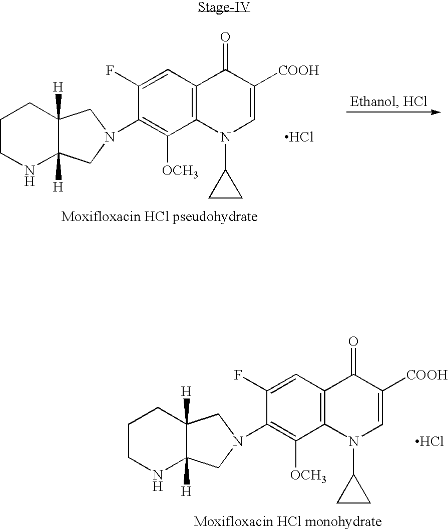

### BRIEF DESCRIPTION OF THE DRAWINGS

FIG. 1: X-ray diffraction pattern of the (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O) borate.

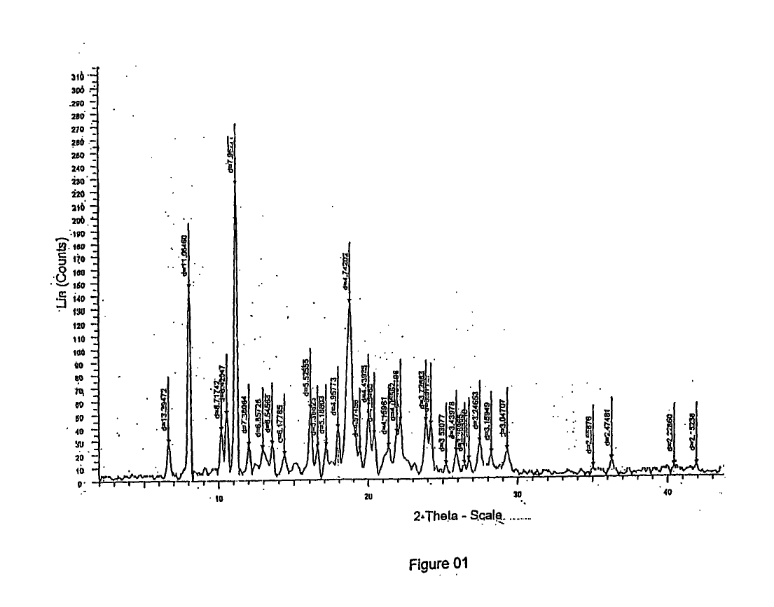

FIG. 2: FTIR spectrum of the (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate

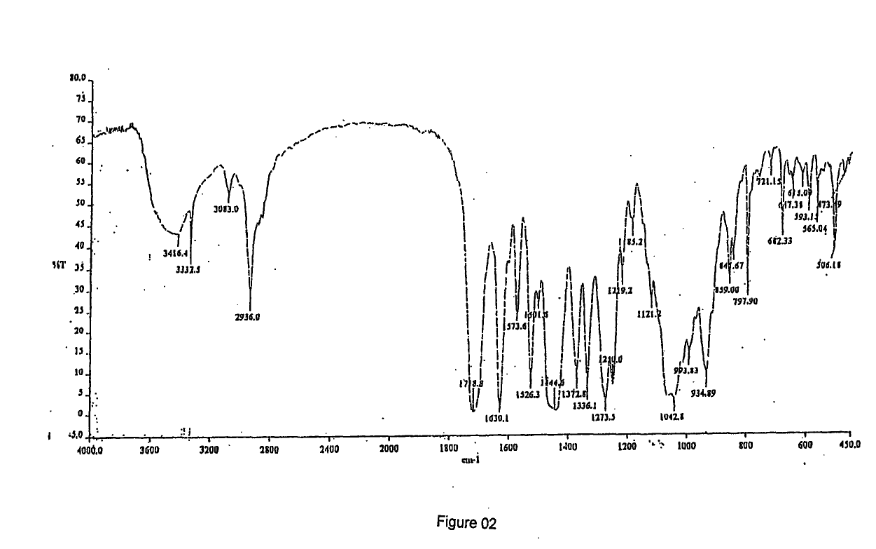

FIG. 3: NMR spectrum of the (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate

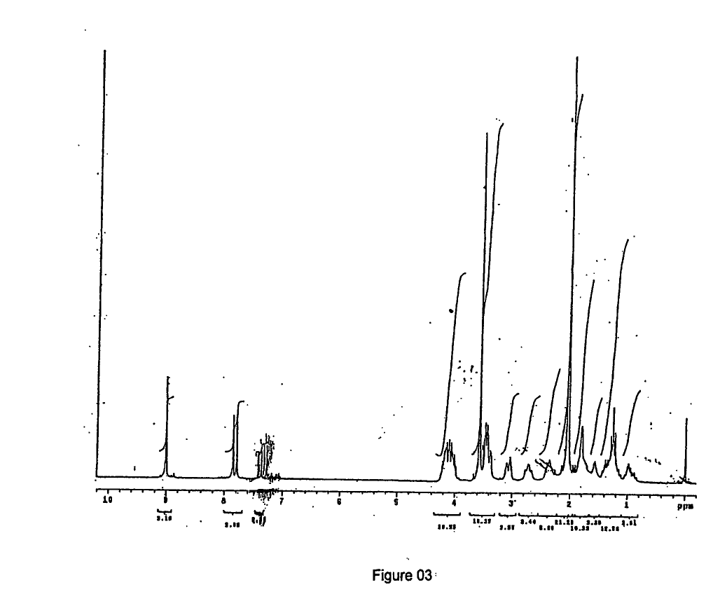

FIG. 4: FTIR spectrum of the Moxifloxacin hydrochloride psuedohydrate

FIG. 5: X-ray diffraction pattern of the Moxifloxacin hydrochloride psuedohydrate

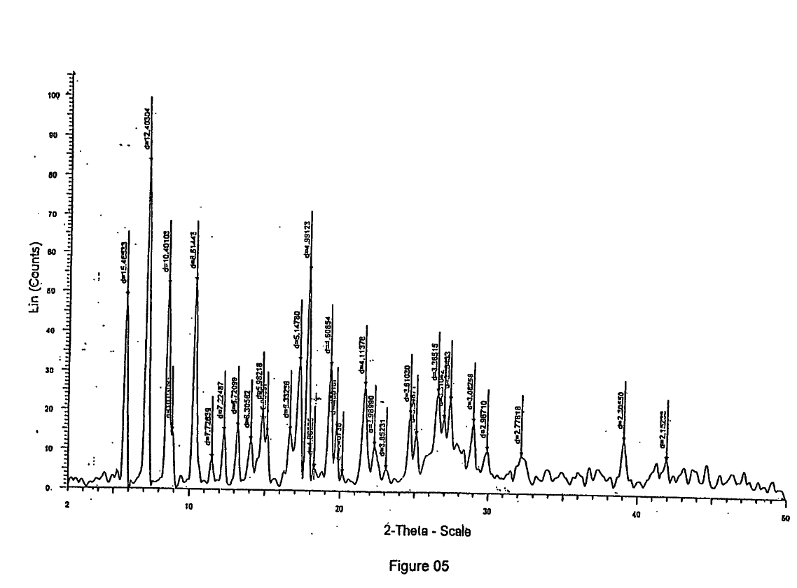

FIG. 6: FTIR spectrum of the Moxifloxacin hydrochloride anhydrous

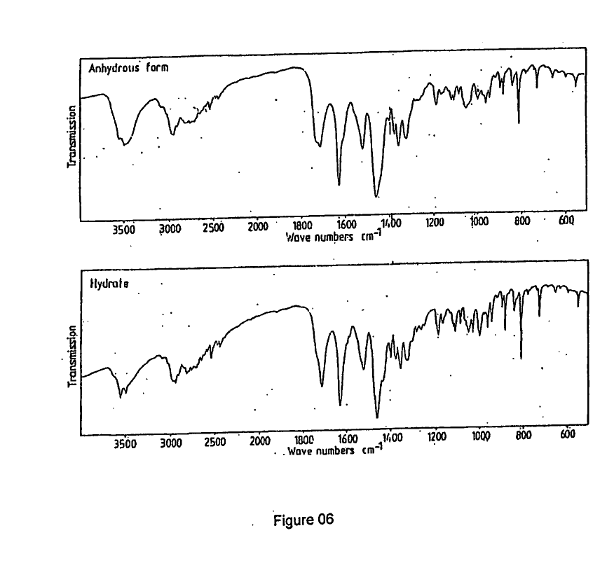

FIG. 7: X-ray diffraction pattern of the Moxifloxacin hydrochloride anhydrous

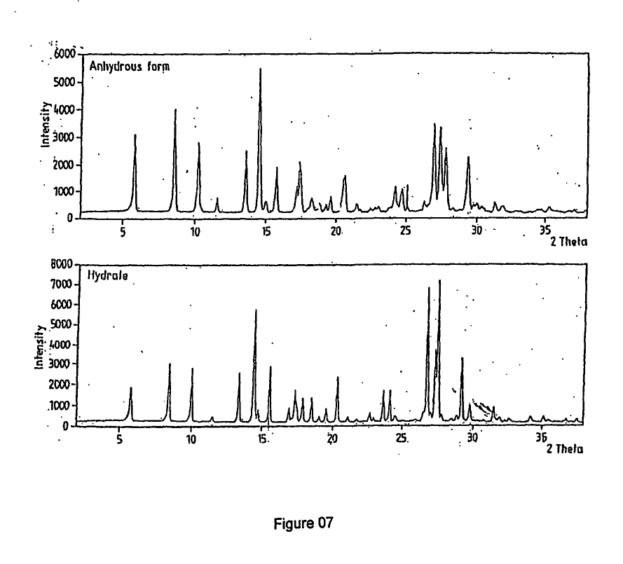

FIG. 8: FTIR spectrum of the Moxifloxacin hydrochloride monohydrate

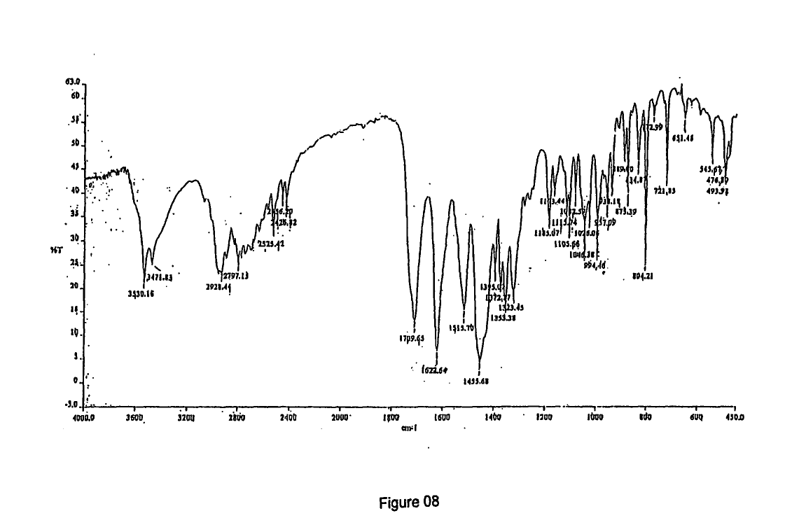

FIG. 9: X-ray diffraction pattern of the Moxifloxacin hydrochloride monohydrate

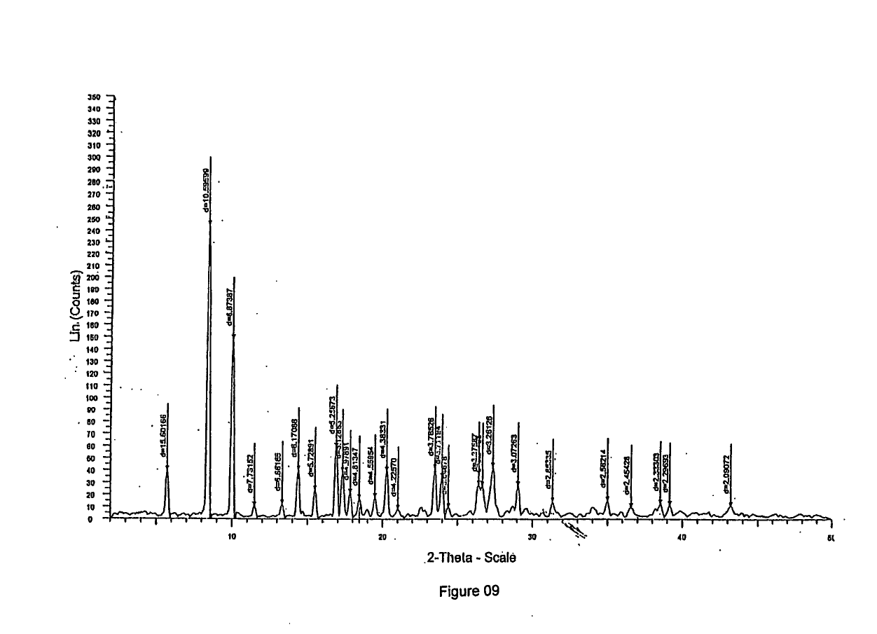

### DETAILED DESCRIPTION OF THE INVENTION

The process of the present invention comprises steps as:
Reacting ethyl 1-cyclopropyl-6,7-difluoro-8-methoxy-4-oxo-1,4-dihydro-3-quinoline carboxylate with a mixture of boric acid and acetic anhydride at temperature above 50° C. without the use of catalyst
Separating (1-cyclopropyl-6,7-difluoro-8-methoxy-4-oxo-1,4-dihydro-3-quinoline carboxylic acid-O³,O⁴)bis(acyloxy-O) borate by cooling to low temperature followed by dilution with water
Isolating and drying the (1-cyclopropyl-6,7-difluoro-8-methoxy-4-oxo-1,4-dihydro-3-quinoline carboxylic acid-O³,O⁴)bis(acyloxy-O)borate
Condensing (1-cyclopropyl-6,7-difluoro-8-methoxy-4-oxo-1,4-dihydro-3-quinoline carboxylic acid-O³,O⁴)bis(acyloxy-O)borate with (S,S)-2,8-diazabicyclo[4.3.0]nonane in presence of base(s) in organic polar solvent(s)
Optionally Isolating the novel intermediate after completion of reaction (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate
Optionally without isolating the intermediate, directly proceeding to the preparation of Moxifloxacin hydrochloride by reaction with hydrochloric acid in a solvent
Isolating and drying the Moxifloxacin hydrochloride pseudohydrate
Optionally treating the Moxifloxacin hydrochloride pseudohydrate with hydrochloric acid in ethanol to get Moxifloxacin hydrochloride monohydrate

The prepared 1-cyclopropyl-6,7-difluoro-8-methoxy-4-oxo-1,4-dihydro-3-quinoline carboxylic acid-O³,O⁴)bis(acyloxy-O)borate is a hydrate and the novel intermediate (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate is anhydrous, characterized by chemical analysis NMR, IR spectrum and XRD.

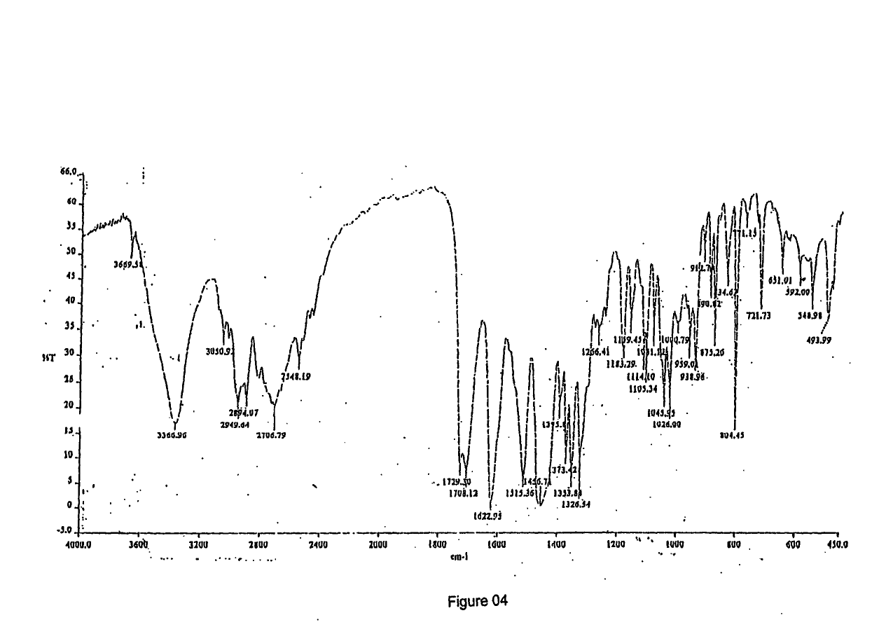

Moxifloxacin hydrochloride pseudohydrate prepared by the process of this invention exhibits some novel characteristics such as water content varying from 0.5% to 1.0%, and high hygroscopic nature. However the XRD data and IR patterns of the pseudohydrate as prepared remains substantially unaltered as illustrated in FIGS. 4 & 5.

The starting materials ethyl 1-cyclopropyl-6,7-difluoro-8-methoxy-4-oxo-1,4-dihydro-3-quinoline carboxylate and [S,S]-2,8-diaza bicyclo[4.3.0]nonane are prepared by literature reported methods.

Acetic anhydride is heated to about 70° C., and boric acid is added in lots. The reaction mass is stirred for about 1 hr to about 2 hrs at temperatures of about 70° C.-about 125° C., preferably at about 110° C. to-about 120° C., cooled to temperature of about 60° C.-about 100° C., preferably to about 70° C. To this mixture, ethyl(1-cyclopropyl-6,7-difluoro-8-methoxy-4-oxo-1,4-dihydro-3-quinoline carboxylate is added, the temperature raised to about 90° C.-about 120° C., preferably to about 100° C. to about 110° C. and mixed for about 1 hr to about 5 hrs preferably for about 1 hr. The reaction mass is cooled to temperature below 35° C., preferably to about 0° C.-about 20° C., preferably to about 0° C. followed by addition of cold water and then mixed for about 1 to about 4 hrs. The product formed is separated by conventional means, washed with water and dried to obtain 1-cyclopropyl-6,7-difluoro-8-methoxy-4-oxo-1,4-dihydro-3-quinoline carboxylic acid-O³,O⁴)bis(acyloxy-O)borate.

(1-Cyclopropyl-6,7-difluoro-8-methoxy-4-oxo-1,4-dihydro-3-quinoline carboxylic acid-O³,O⁴)bis(acyloxy-O)borate is suspended in organic polar solvents preferably DMSO, DMF, acetonitrile, ethanol and mixed with [S,S]-2,8-Diaza bicyclo[4.3.0]nonane in presence of organic, inorganic base(s) preferably triethyl amine, DBU, diisopropylethyl amine, potassium carbonate at temperatures about 20° C.-about 120° C., preferably at about 60° C.-about 80° C. for about 1 hr to about 6 hrs. After the completion of reaction the reaction mass is cooled. The novel intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate is isolated by removal of solvent under vacuum below 60° C. preferably at about 40° C.-45° C. followed by addition of the hydrocarbons preferably hexane, heptane, cyclohexane, methyl cyclohexane, mixed for about 2 hrs, the product is filtered and dried.

Alternatively it is possible to proceed to the preparation of Moxifloxacin hydrochloride pseudohydrate without the isolation of the intermediate (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate as follows:

The reaction mass is diluted with short chain alcohol, with an optional step of the removal of insolubles (if any), adjusting the pH of the reaction mass to acidic with hydrochloric acid at temperatures below 35° C. preferably in the range of about 20° C. to about 25° C. and stirred for about 2 to about 6 hrs. The alcohol is selected from C-1 to C-4 alcohols preferably methanol and/or ethanol. The pH is adjusted to below 2.0 preferably between below 0.5 and cooled to below 15° C. preferably between about 0° C. to about 5° C. and maintained for about 2 to about 6 hrs. The product is separated and dried to obtain Moxifloxacin hydrochloride pseudohydrate.

In another embodiment of the invention, the isolated intermediate (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O) borate is converted directly to Moxifloxacin hydrochloride by dissolving in short chain alcohol preferably ethanol, methanol, removing the insolubles if any, adjusting pH to below 2.0 preferably to below 0.5 with hydrochloric acid and maintaining for about 1 hr to about 4 hrs preferably for about 2 hrs at temperatures in the range of about 20° C. to about 25° C. After completion of reaction, the reaction mass cooled to below 15° C. preferably in the range of about 0° C. to about 5° C. and maintained for about 2 to about 6 hrs. The product is separated and dried to obtain Moxifloxacin hydrochloride pseudohydrate.

Moxifloxacin hydrochloride pseudohydrate upon mixing with hydrochloric acid in presence of ethanol at temperature yields Moxifloxacin hydrochloride monohydrate.

The invention is now illustrated with a few non-limiting examples.

### EXAMPLE-I

Stage-1: Preparation of 1-cyclopropyl-6,7-difluoro-8-methoxy-4-oxo-1,4-dihydro-3-quinoline carboxylic acid-O³,O⁴)bis(acyloxy-O)borate

Acetic anhydride (175 g) is heated to 70° C. and boric acid (30 g) is slowly added lot wise in a temperature range of 70° C. to 90° C. The temperature is then raised, maintained under reflux for 1 hr followed by cooling to about 70° C. Ethyl-1-cyclopropyl-6,7-difluoro-8-methoxy-4-oxo-1,4-dihydro-3-quinoline carboxylate (100 g) is added under stirring. The temperature is then raised and maintained for 1 hr in the range of 100° C. to 105° C. The reaction mass is cooled to 0° C., chilled water (400 ml) is added slowly followed by cold water (600 ml) at temperature 0° C. to 5° C. and maintained for 2 hrs at 0° C. to 5° C. The product which is a boron acetate complex is filtered, washed with water (500 ml) and dried at 55° C. to 60° C. under vacuum to constant weight.

The dry wt is 130.0 g corresponding to yield of 95.2%.

Stage-2: Preparation of (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate

The boron acetate complex (130 g) prepared in stage 1 is suspended in acetonitrile (650 ml), and [S,S]-2,8-diazabicyclo[4.3.0]nonane (47 g) and triethyl amine (72.9 g) are added. The temperature is raised to reflux and maintained for 1 hr. at reflux, followed by cooling to about 40° C. The solvent is removed under vacuum at temperature below 40° C., and n-hexane (200 ml) is added. After maintaining the reaction mass for 1 hr at room temperature the product is isolated by filtration followed by washing of the wet cake with n-hexane. The product is dried at about 45° C. to about 50° C. to constant weight.

Dry wt of the novel intermediate is 117.0 g corresponding to yield of 71.5%.

Elemental analysis: C: 56.42%, H: 5.62%, N: 7.76% and the calculated values for the intermediate, formula C₂₅H₂₉BFN₃O₈ C: 56.6%, H: 5.47%, N: 7.92%

IR Spectrum (KBr, cm⁻¹): 3415, 3332, 2936, 1718, 1630, 1573, 1526, 1445, 1273, 1042, 935, 860, 798, 682

¹H NMR (200 MHz, CDCl₃, ppm): 9.00 (1H), 7.82 (1H), 4.12 (4H), 3.57 (3H), 3.43 (4H), 3.07 (2H), 2.75 (2H), 2.4 (1H), 2.1 (6H), 1.84 (2H), 1.6 (1H), 1.31 (2H)

Mass Spectrum (M⁺): 530.3 [M⁺H], 470.2 [M⁺-CH₃COOH], 428.2 [M⁺-(CH₃CO)₂O, 100%], 402.2, 388.2

Stage-3: Preparation of Moxifloxacin Hydrochloride Pseudohydrate

The intermediate (117 g) prepared stage-2 is dissolved in ethanol (600 ml) by stirring for about 30 min. at room temperature and the insolubles if any are filtered off. pH of the filtrate is adjusted to about 0.5 by addition of hydrochloric acid at room temperature and maintained for 2 hrs. The reaction mass is cooled, and maintained for two hrs, at about 0° C. to about 5° C. The product is filtered, washed with chilled ethanol (50 ml) and dried at about 50° C. to about 55° C. till constant weight.

The dry weight of the Moxifloxacin hydrochloride pseudohydrate is 87.5 g corresponding to yield of 91.0%. Water content of the product by KF is 0.64% w/w.

X-ray diffraction pattern data are given in Table-1

### EXAMPLE-II

Stage-2: Preparation of Moxifloxacin Pseudohydrate with out Isolating (4aS-Cis)-1-Cyclopropyl-7-(2,8-diazabicyclo[4.3.0]non-8-yl)-6-fluoro-8-methoxy-4-oxo-1,4-dihydro-3-quinolinecarboxylic acid-O³,O⁴)bis(acyloxy-O) borate

The boron acetate complex (130 g) prepared in stage-1 of Example-1 is suspended in acetonitrile (650 ml) and [S,S]-2,8-Diazabicyclo[4.3.0]nonane (47 g) & triethyl amine (72.9 g) are added. Temperature of the reaction mass is raised to reflux, maintained for 1 hr. at reflux and cooled to room temperature. Methanol (600 ml) is added and maintained for 30 min at room temperature to obtain a clear solution. The solution is filtered to remove insolubles if any and pH of the filtrate is adjusted to about 0.5 with hydrochloric acid (57.5 g). The reaction mass is maintained for 2 hrs at temperature in the range of about 20° C. to about 25° C., cooled to 0° C. followed by maintaining the reaction mass at about 0° C. to about 5° C. for 2 hrs. The product is filtered, washed with methanol (50 ml) and dried at about 50° C. to 55° C. until constant weight.

Dry wt of the Moxifloxacin hydrochloride pseudohydrate is 88 g corresponding to yield of 68.7%.

### EXAMPLE-III: Preparation of Moxifloxacin Hydrochloride Monohydrate

Moxifloxacin hydrochloride (50 g) prepared as above is suspended in a mixture of ethanol (250 ml) and hydrochloric acid (25 ml). Raised the temperature, maintained for two hrs at 40° C. to 45° C. followed by cooling to about 25° C. The product is filtered and dried under vacuum at 50-55° C. until become constant weight.

Dry wt of Moxifloxacin hydrochloride monohydrate is 46 g corresponding to yield of 90.5%.

The IR spectral data and XRD pattern are identical with available Moxifloxacin hydrochloride monohydrate.

TABLE 1

| | | | |
|---|---|---|---|
| S. No | PSEUDOHYDRATE | MONO HYDRATE | ANHYDROUS |

FTIR PEAKS OF MOXIFLOXACIN HYDROCHLORIDE

| | | | |
|---|---|---|---|
| 1 | 3669 | 3530 | 3527 |
| 2 | 3357 | 3472 | 3469 |
| 3 | 2950 | 2925 | 2929 |
| 4 | 2894 | 2525 | 2524 |
| 5 | 2548 | 2456 | |
| 6 | 1730 | 2427 | 2427 |
| 7 | 1708 | 1709 | 1709 |
| 8 | 1623 | 1623 | 1621 |
| 9 | 1515 | 1516 | 1512 |
| 10 | 1456 | 1456 | 1452 |
| 11 | 1373 | 1395 | |
| 12 | 1354 | 1372 | 1371 |
| 13 | 1326 | 1353 | 1353 |
| 14 | 1183 | 1185 | 1186 |
| 15 | 1046 | 1046 | 1048 |
| 16 | 1028 | 994 | 994 |
| 17 | 938 | 938 | 938 |
| 18 | 875 | 875 | 709 |
| 19 | 835 | 835 | 834 |
| 20 | 804 | 804 | 804 |
| 21 | 722 | 722 | 722 |

XRD PEAKS OF MOXIFLOXACIN HYDROCHLORIDE

| | | | |
|---|---|---|---|
| 1 | 5.8 | 5.7 | 5.8 |
| 2 | 7.2 | 8.3 | 8.6 |
| 3 | 8.6 | 10 | 10.2 |
| 4 | 10.4 | 11.4 | 11.5 |
| 5 | 12.4 | 13.3 | 13.5 |
| 6 | 13.3 | 14.3 | 14.3 |
| 7 | 14.6 | 15.5 | 15 |
| 8 | 14.9 | 16.9 | 15.7 |
| 9 | 15.2 | 17.3 | 17.2 |
| 10 | 16.7 | 17.8 | 17.4 |
| 11 | 17.3 | 18.4 | 18.2 |
| 12 | 17.9 | 19.5 | 18.8 |
| 13 | 18.7 | 20.2 | 19.2 |
| 14 | 19.8 | 23.5 | 19.5 |
| 15 | 21.7 | 24 | 20.6 |
| 16 | 22.4 | 26.4 | |
| 17 | 24.7 | 26.6 | 21.5 |
| 18 | 25.2 | 27.3 | 22.5 |
| 19 | 25.8 | 29 | |
| 20 | 26.6 | 31.3 | |
| 21 | 27 | 35 | |
| 22 | 27.4 | 36.6 | |
| 23 | 27.9 | 38.6 | |
| 24 | 28.4 | 39.2 | |
| 25 | 29 | 43.2 | |
| 26 | 30 | | |
| 27 | 31.6 | | |
| 28 | 32.3 | | |
| 29 | 35 | | |
| 30 | 37.6 | | |
| 31 | 39.1 | | |
| 32 | 41.3 | | |
| 33 | 41.9 | | |
| 34 | 43.9 | | |

1. A process for the preparation of Moxifloxacin hydrochloride monohydrate comprising steps
Treating (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid (-O³,O⁴)bis(acyloxy-O) borate with hydrochloric acid in a solvent
Isolating and drying the Moxifloxacin hydrochloride
Treating the Moxifloxacin hydrochloride with hydrochloric acid in ethanol to get Moxifloxacin hydrochloride monohydrate

2. A process as claimed in claim-1, wherein hydrochloric acid is gaseous or aqueous or dissolved in a solvent

3. A process as claimed in claim-1, wherein the solvent used is a short chain alkanol

4. A process as claimed in claim-3, wherein the short chain alkanol is preferably methanol, ethanol and isopropanol

5. Moxifloxacin hydrochloride which is characterized by an infrared absorption comprising bands at 3669, 3357, 2950, 2894, 2548, 1730, 1708, 1623, 1515, 1456, 1373, 1354, 1326, 1183, 1046, 1028, 938, 875, 835, 804 and 722 cm⁻¹

6. Moxifloxacin hydrochloride which is characterized by a powder X-ray diffraction pattern comprising peaks at about 5.8, 7.2, 8.6, 10.4, 12.4, 13.3, 14.6, 14.9, 15.2, 16.7, 17.3, 17.9, 18.7, 19.8, 21.7, 22.4, 24.7, 25.2, 25.8, 26.6, 27.0, 27.4, 27.9, 28.4, 29.0, 30.0, 31.6, 32.3, 35.0, 37.6, 39.1, 41.3, 41.9 and 43.9±0.2 degrees two-theta.

7. Crystalline (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate

8. As claimed in claim-8 the novel intermediate which is characterized by an infrared absorption comprising bands at 3415, 3332, 2936, 1718, 1630, 1573, 1526, 1445, 1273, 1042, 935, 860, 798, 682 cm⁻¹

9. A process for the preparation of a novel intermediate (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis (acyloxy-O)borate comprising:
Reacting ethyl 1-cyclopropyl-6,7-difluoro-8-methoxy-4-oxo-1,4-dihydro-3-quinoline carboxylate with a mixture of boric acid and acetic anhydride at temperature above 50° C. without the use of catalyst
Precipitating (1-Cyclopropyl-6,7-difluoro-8-methoxy-4-oxo-1,4-dihydro-3-quinoline carboxylic acid-O³,O⁴)bis(acyloxy-O)borate by cooling to low temperature followed by diluting with water
Isolating and drying the (1-cyclopropyl-6,7-difluoro-8-methoxy-4-oxo-1,4-dihydro-3-quinoline carboxylic acid-O³,O⁴)bis(acyloxy-O)borate
Condensing (1-Cyclopropyl-6,7-difluoro-8-methoxy-4-oxo-1,4-dihydro-3-quinoline carboxylic acid-O³,O⁴)bis(acyloxy-O)borate with (S,S)-2,8-Diazabicyclo[4.3.0]nonane in presence of base(s) in organic polar solvent(s)
Crystallizing (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate
Isolating and drying of (4aS-Cis)-1-cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate

10. The process as claimed in claim 9, wherein the temperature for the reaction of ethyl 1-cyclopropyl-6,7-difluoro-8-methoxy-4-oxo-1,4-dihydro-3-quinoline carboxylate with the mixture of boric acid and acetic anhydride is in the range of 90° C. to 120° C.

11. The process as claimed in claim 9, wherein the organic polar solvents is selected from acetonitrile or DMSO or DMF.

12. The process as claimed in claims 9, wherein the base(s) used is organic or inorganic base

13. The process as claimed in claims 12, wherein the organic base is selected from triethylamine or diisopropyl ethylamine or DBU

14. The process as claimed in claims 12, wherein the inorganic base is potassium carbonate

15. The process as claimed in claim 9, wherein the temperature for the condensation reaction is in the range of 30° C. to 100° C., preferably from 60° C. to 80° C.

16. The process as claimed in claim 9, wherein the crystallization of (4aS-Cis)-1-Cyclopropyl-7-(2,8-diazabicyclo[4.3.0]non-8-yl)-6-fluoro-8-methoxy-4-oxo-1,4-dihydro-3-quinoline carboxylic acid-O³,O⁴)bis(acyloxy-O)borate is carried out by removal of solvent and adding a second solvent

17. The process as claimed in claim 16, wherein the second solvent is selected from hydrocarbons of C-5 to C-7

18. The process as claimed in claim 17, wherein the hydrocarbon is alkanes, cycloalkanes or mixtures thereof

19. The process as claimed in claim 17, wherein the hydrocarbon is n-hexane, n-heptane, cyclohexane, methyl cyclohexane or mixtures thereof.
